(19) United States
(12) Patent Application Publication
(10) Pub. No.: US 2024/0255442 A1
(43) Pub. Date: Aug. 1, 2024

(54) SYSTEM AND METHOD FOR DETERMINING AT LEAST A PROPERTY OF MULTIPHASE FLUID

(71) Applicant: Schlumberger Technology Corporation, Sugar Land, TX (US)

(72) Inventors: Cheng-Gang XIE, Singapore (SG); Elodie MARQUINA GUINOIS, Clamart (FR); Yu Ke LIM, Singapore (SG)

(21) Appl. No.: 18/560,689

(22) PCT Filed: May 18, 2022
(86) PCT No.: PCT/US2022/029762

§ 371 Date: Nov. 14, 2023

Related U.S. Application Data
(60) Provisional application No. 63/190,044, filed on May 18, 2021.

(51) Int. Cl. G01N 22/00; G01F 1/44; G01N 33/18

(52) U.S. Cl. CPC G01N 22/00; G01F 1/44; G01N 33/18

(57) ABSTRACT

A system can include a conduit; a water analysis sensor arranged on the conduit that measures at least a property of multiphase fluid in the conduit; at least a pair of microwave antennas, where the pair of microwave antennas includes a first antenna that transmits a signal into the multiphase fluid in the conduit and a second antenna that receives at least a portion of the signal as transmitted into the multiphase fluid in the conduit; and a processing system to: determine at least a property of the liquid water phase based on output of the water analysis sensor, where the at least a property includes one or more of a water salinity and a water liquid ratio, determine one or more of a mixture permittivity and a mixture conductivity of the multiphase fluid based on output of at least one antenna, and determine a gas hold-up of the multiphase fluid based at least in part on one or more of the mixture permittivity and the mixture conductivity.

### RELATED APPLICATIONS

This application claims priority to and the benefit of a U.S. Provisional Application having Ser. No. 63/190,044, filed 18 May 2021.

### TECHNICAL FIELD

This disclosure relates to multiphase flow measurement systems that may be used standalone or with multiphase flow meters at downhole, surface, or subsea locations.

### BACKGROUND

Wells are generally drilled into subsurface rocks to access fluids, such as hydrocarbons or geothermal resources, stored in subterranean formations. The subterranean fluids can be produced from these wells through various techniques. Operators may want to know certain characteristics of produced fluids to facilitate efficient and economic exploration and production. For example, operators may want to know flow rates of produced fluids. These produced fluids can include multiphase fluids (e.g., consider phases that may include water, oil, and gas or steam vapor), which can make measurement of the flow rates more complex.

Various systems can be used to determine flow rates for multiphase fluids. In some systems, multiphase fluids are separated into their constituent phases and these phases are then individually measured by using single-phase flow meters to determine flow rates. Other systems include multiphase flow meters that can be used to measure flow rates of multiphase fluids without separation. These multiphase flow meters may be smaller and lighter than those with separator-based single-phase flow meters. A system with the ability to measure flow rates without separation may be desirable in some instances. Some separator-based systems and multiphase flow meter systems may also be used to determine certain other fluid characteristics of interest.

It is also desirable to determine properties of a multiphase mixture, such as the presence, fraction, and salinity of water in a multiphase mixture, and the water-in-liquid ratio, steam quality, gas hold-up as this provides information about produced fluid and/or injected water in the mixture, about the (subsea) flow-assurance measures needed to prevent hydrate formation and/or pipeline corrosion, etc. One or more of such conditions may affect one or more other measurements being made on a multiphase mixture.

With reference to multiphase flow in pipes, hold-up is the fraction of a particular fluid present in an interval of pipe. In multiphase flow, each fluid can move at a different speed due to various factors (e.g., different gravitational forces and other factors), for example, with a heavier phase moving slower, or being more held up, than the lighter phase. The holdup of a particular fluid is not the same as the proportion of the total flow rate due to that fluid, also known as its cut. Determination of in-situ flow rates demands measurement of holdup and velocity of each fluid. The holdup ratio is the ratio of the holdups of two fluids, and is sometimes used as a parameter to express the phenomenon.

A published U.S. patent application (US 2018/0298748 A1, published 18 Oct. 2018, referred to herein as the '748 application) describes a method for determining the flow rate of formation water and/or the total rate of produced water in a gas well. More particularly, the method can be implemented using a system to determine the flow rates of gas, condensate, and water in gas production using a non-gamma flowmeter based on venturi differential pressure (and pressure and temperature) measurements, and the water salinity measured by a water analyzer (such as a microwave reflection conductivity/salinity probe) installed flush with the outlet pipe.

The system of the '748 application as well as various other systems may experience performance issues when trying to make flow measurements for a fluid having a high Gas Volume Fraction (GVF), i.e., the ratio of gas volumetric flow rate to the total volumetric flow rate of all fluids. Such flow measurements are referred to herein as high-GVF flow measurements. High-GVF flow measurements can be quantified using a percentage, for example, when the GVF of the fluid is above 90% or above 95%. For example, a high-GVF can be defined as being greater than 90% or greater than 95%.

### SUMMARY

A system can include a conduit; a water analysis sensor arranged on the conduit that measures at least a property of multiphase fluid in the conduit, where the multiphase fluid includes at least a gaseous phase and a liquid water phase; at least a pair of microwave antennas, where the pair of microwave antennas includes a first antenna that transmits a signal into the multiphase fluid in the conduit and a second antenna that receives at least a portion of the signal as transmitted into the multiphase fluid in the conduit; and a processing system that includes a processor, memory accessible to the processor, and processor-executable instructions stored in the memory and executable by the processor to cause the processing system to: determine at least a property of the liquid water phase based on output of the water analysis sensor, where the at least a property includes one or more of a water salinity and a water liquid ratio, determine one or more of a mixture permittivity and a mixture conductivity of the multiphase fluid based on output of the second antenna, and determine a gas hold-up of the multiphase fluid based at least in part on one or more of the mixture permittivity and the mixture conductivity.

A method can include receiving flowing multiphase fluid in a conduit; acquiring measurements with respective sensors relative to one or more properties of the multiphase fluid, where the sensors include at least a water analysis sensor and a pair of microwave antennas, where the pair of microwave antennas includes a first antenna for transmitting a signal and a second antenna for receiving at least a portion of the signal; determining at least a property of a liquid water phase of the multiphase fluid based on an acquired water analysis sensor measurement, where the at least a property includes one or more of a water salinity and a water liquid ratio; determining one or more of a mixture permittivity and a mixture conductivity of the multiphase fluid based on the at least a portion of the signal received by the second antenna of the pair; and determining a gas hold-up of the multiphase fluid based at least in part on one or more of the mixture permittivity and the mixture conductivity.

One or more computer-readable storage media can include processor-executable instructions executable by a processor to instruct a system to: acquire measurements with respective sensors relative to one or more properties of multiphase fluid flowing in a conduit, where the sensors include at least a water analysis sensor and a pair of microwave antennas, where the pair of microwave antennas includes a first antenna for transmitting a signal and a second antenna for receiving at least a portion of the signal; determine at least a property of a liquid water phase of the multiphase fluid based on an acquired water analysis sensor measurement, where the at least a property includes one or more of a water salinity and a water liquid ratio; determine one or more of a mixture permittivity and a mixture conductivity of the multiphase fluid based on the at least a portion of the signal received by the second antenna of the pair; and determine a gas hold-up of the multiphase fluid based at least in part on one or more of the mixture permittivity and the mixture conductivity.

### BRIEF DESCRIPTION OF THE DRAWINGS

FIG. 1 is a cross-sectional cutaway view of an example of a system.

FIG. 2 is a cross-sectional cutaway view of an example of a system.

FIG. 3A and FIG. 3B are side views of an example of a system.

FIG. 4 is a diagram of an example of a system.

FIG. 5A and FIG. 5B are perspective views of an example of a portion of a system.

FIG. 6 is a block diagram of examples of components and methods.

FIG. 7 is a block diagram of examples of components and methods.

FIG. 8 is a block diagram of an example of a method.

FIG. 9 is a series of block diagrams of examples of systems.

### DETAILED DESCRIPTION

The following description includes the best mode presently contemplated for practicing the described implementations. This description is not to be taken in a limiting sense, but rather is made merely for the purpose of describing the general principles of the implementations. The scope of the described implementations should be ascertained with reference to the issued claims.

As an example, a system can provide for implementation of a method for performing flow measurements, which can include high-GVF flow measurements. In such an example, flow measurements may be obtained at the exit of a wellbore in which multiphase fluid is produced.

As an example, a system can include various components such as, for example, a conduit for flowing the multiphase fluid; a water analysis sensor arranged on the conduit for measuring at least a property of the fluid; at least a pair of microwave antennas, where the microwave antennas include a first antenna and a second antenna arranged on the conduit, where the first antenna transmits a signal and the second antenna receives the signal; and a processing system that can include circuitry that can determine (i) at least a property of the liquid water phase based on the water analysis sensor measurement, where the at least a property includes at least one of a water salinity, a water permittivity, a water conductivity and water liquid ratio, (ii) at least one of a mixture permittivity (such as a complex mixture permittivity) and a mixture conductivity of the fluid based on the signal received by the second antenna of the probe, and (iii) a gas hold-up of the fluid based on the mixture permittivity and/or conductivity and the water property.

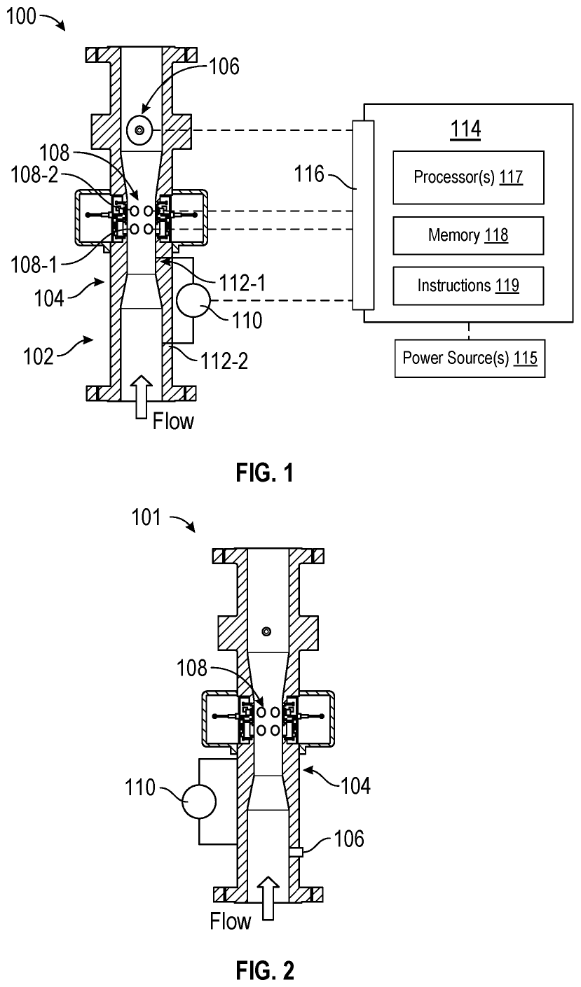

Several example embodiments of a system with features for performing measurements, including high-GVF measurements, are shown in FIGS. 1-3 and described below.

FIG. 1 shows an example of a system 100 according to a first embodiment. The system includes a conduit 102 having a flow restriction 104 (e.g., a Venturi), and a water analysis sensor 106 arranged in the conduit 102 and disposed downstream (e.g., in an outlet region after an end of the flow restriction), and at least a pair 108 of microwave antennas also arranged in the conduit 102. As shown in the example of FIG. 1, two pairs 108-1 and 108-2 of microwave antennas can be disposed in the throat of the flow restriction 104. The system 100 can also include a differential pressure (DP) sensor 110 for measuring a pressure difference between two locations 112-1 and 112-2, situated respectively upstream the flow restriction 104 and between opposing ends of the flow restriction 104 (e.g., at or proximate an inlet and in a throat of a Venturi).

As indicated by a large arrow, multiphase fluid can flow into the conduit 102. The multiphase fluid is a fluid that includes at least a liquid phase and a gas phase. It can include a liquid phase, for instance a water liquid phase—such as in a geothermal setup (with a gas phase being water vapor or steam)—or a plurality of liquid phases, for example, consider a water liquid phase and an oil liquid phase in a 3-phase fluid that is generally obtained when producing a hydrocarbon well.

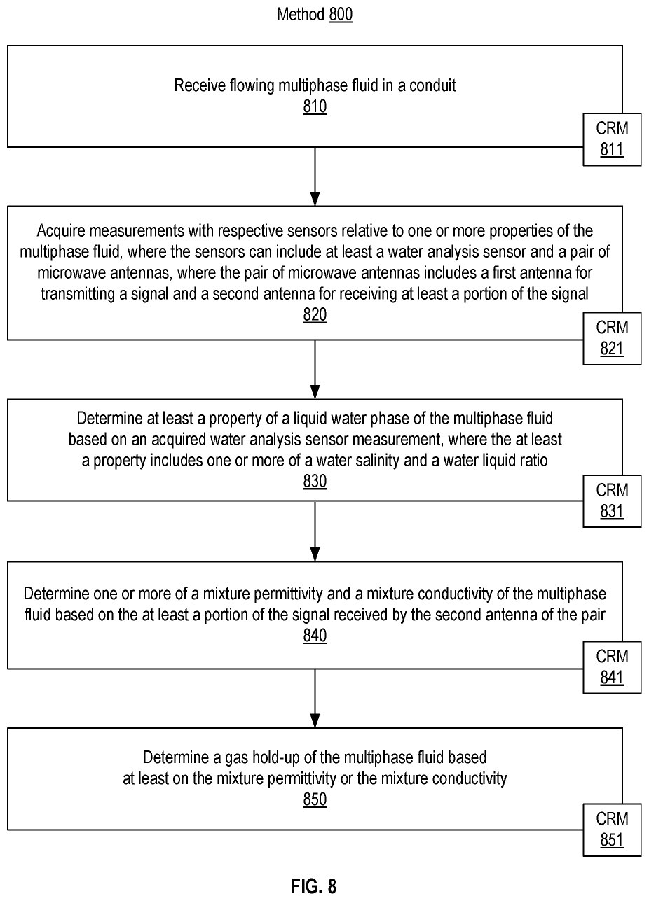

As shown in the example of FIG. 1, the system 100 can also include a processing system 114 that can include one or more processors 117, operatively coupled to each of the sensors for acquiring data from the sensors and determining one or more parameters, as explained below. As shown, the processing system 114 can include one or more interfaces 116, which can be wired and/or wireless interfaces that can operatively couple to sensors for acquisition of data and/or for control of one or more of the sensors. As an example, the processing system 114 can be operatively coupled to one or more power sources 115, which may include a grid power source, a battery power source, a capacitor power source, a turbine power source (e.g., gas combustion turbine, fluid driven turbine, etc.), a solar power source, etc. As an example, one or more sensors may be directly and/or indirectly coupled to one or more of the one or more power sources 115. As an example, the processing system 114 can include the one or more processors 117 (e.g., cores, etc.) and memory 118 that is processor-accessible. In such an example, the memory 118 can be a memory device that is a physical device that is non-transitory and not a carrier wave. In such an example, the memory device can be referred to as a processor-readable storage medium or a computer-readable storage medium. Such a memory device can store processor-executable instructions 119 (e.g., computer-executable instructions) that can be executed by the processing system 114 to cause the processing system 114 to perform various actions. For example, consider a method that includes various actions related to operation of the system 100 (e.g., one or more sensors, one or more antennas, etc.), determinations based on data generated by the system 100, etc. (see, e.g., FIG. 8, etc.).

As an example, the processing system 114 may include network circuitry, for example, consider network circuitry that is part of the one or more interfaces 116. As an example, the processing system 114 may be a gateway that can include, for example, one or more features of the AGORA gateway (e.g., consider one or more processors, memory, etc., which may be deployed as a “box” that can be locally powered and that can communicate locally with equipment via one or more interfaces). As an example, one or more pieces of equipment may include computational resources that can be akin to those of an AGORA gateway or more or less than those of an AGORA gateway. As an example, an AGORA gateway may be a network device with various networking capabilities.

As an example, a gateway can include one or more features of an AGORA gateway (e.g., v.202, v.402, etc.) and/or another gateway. For example, consider features such as an INTEL ATOM E3930 or E3950 dual core with DRAM and an eMMC and/or SSD. Such a gateway may include a trusted platform module (TPM), which can provide for secure and measured boot support (e.g., via hashes, etc.). A gateway may include one or more interfaces (e.g., Ethernet, RS485/422, RS232, etc.). As to power, a gateway may consume less than about 100 W (e.g., consider less than 10 W or less than 20 W). As an example, a gateway may include an operating system (e.g., consider LINUX DEBIAN LTS or another operating system). As an example, a gateway may include a cellular interface (e.g., 4G LTE with global modem/GPS, 5G, etc.). As an example, a gateway may include a WIFI interface (e.g., 802.11 a/b/g/n). As an example, a gateway may be operable using AC 100-240 V, 50/60 Hz or 24 VDC. As to dimensions, consider a gateway that has a protective box with dimensions of approximately 10 in×8 in×4 in (e.g., 25 cm×20.3 cm×10.1 cm).

In the system 100, the water analysis sensor 106 can be a sensor that measures a property of multiphase fluid and based on this measurement, the processing system 114 can determine at least a property of the water liquid phase, the property being at least one of the water salinity, the water permittivity, the water conductivity and of the water liquid ratio (e.g., when the fluid is a three-phase fluid).

As an example, a water analysis sensor may be or may include an optical probe. Examples of water analysis sensors using optical probe technology are described in a published PCT application WO 2020/206368 A1, published 8 Oct. 2020 and in a US patent U.S. Pat. No. 9,593,575 B2 granted 14 Mar. 2017, which are both incorporated by reference herein; noting that one or more appropriate sensors that enable derivation of one of the properties of the water liquid phase, for example, as given above, may be utilized.

In an embodiment, the water analysis sensor 106 can be or can include an electromagnetic sensor such as a microwave reflection probe that measures a liquid-rich mixture (complex) permittivity of the flow in the conduit 102, for example, near a pipe wall of the conduit 102. Such a sensor can be located in the neighborhood of the flow restriction 104 to help ensure that the near-wall flow is liquid-rich. To this end, such a sensor may include a liquid enrichment feature. Some examples of electromagnetic sensors that may be used in the system 100 are described in the '748 application and, for example, in a published PCT application WO 2021/011477 A1, published 21 Jan. 2021, both of which are incorporated herein by reference. As an example, a microwave (open-coaxial) reflection probe structure may be utilized, for example, as described in a US patent (U.S. Pat. No. 9,638,556 B2, granted 2 May 2017), which is herein incorporated by reference.

In the example of FIG. 1, the pair of microwave antennas 108-1 and 108-2 can include a first antenna and a second antenna arranged on the conduit 102. In such an example, the first antenna can transmit a signal and the second antenna can receive at least a portion of the transmitted signal. Such antennas can be magnetic-dipole transmission antennas (such as, for example, aperture-cavity antennas with a magnetic-dipole) and can be utilized to determine a resonance peak frequency of the received signal and/or amplitude-attenuation and phase-shift at one or more frequencies of the received signal. Such parameters of the received signal enable derivation of properties of fluids as the signal has been physically modified (e.g., electrically, magnetically, electromagnetically, etc.) when passing through the fluid. In the example of FIG. 1, a transmitting antenna and a receiving antenna on the conduit 102 can be operatively coupled to the processing system 114 (e.g., electronically coupled via wire, etc.). Some examples of antennas are described in a published US patent application (US 20210372834 A1, published 2 Dec. 2021), which is herein incorporated by reference.

In the example embodiment of FIG. 1, the system 100 includes at least four antennas arranged in one or more 2-transmitter and 2-receiver configurations in the same section of the conduit 102 but at different cross-sections of the conduit 102 to perform compensated transmission measurement(s) that tend to be immune to gain drift. An example of such a mechanism is described in a US patent (U.S. Pat. No. 8,536,883 B2, granted 17 Sep. 2013), which is herein incorporated by reference.

As an example, by combining four signals in a particular manner, it is possible to obtain a measure of phase-shift and amplitude-attenuation which can be substantially independent of factors other than those provided by multiphase fluid travelling through a conduit. Therefore, a more accurate estimate of permittivity and/or conductivity can be obtained where accuracy can be maintained over time, despite drifts which may occur in the gain values of the measurement paths related to both transmitting and receiving antennas.

In the example of FIG. 1, the processing system 114 can provide for various determinations, as explained above, of at least a property (e.g., water salinity, water permittivity, water conductivity and/or water liquid ratio) of a liquid water phase based on a water analysis sensor measurement, as well as a mixture permittivity and/or conductivity of the fluid based on the signals received by a second antenna, where the mixture permittivity and/or conductivity can be determined based on resonance frequency or amplitude attenuation and/or phase shift as will be explained hereinafter. Furthermore, the processing system 114 can enable determinations of a gas hold-up (GHU) of fluid based on the determined mixture permittivity and/or conductivity as well as the water liquid phase property. The processing system 114 may also determine a flow rate of at least one phase of the fluid based on the at least one property of the liquid water phase, on the gas hold-up and on the differential pressure measured across the flow restriction. The processing system 114 can utilize one or more fluid models that provide for obtaining the above-mentioned properties. Some examples of fluid models are explained further below.

FIG. 2 illustrates an example of a system 101, which may be considered to be a variant of the example embodiment of FIG. 1 in which the water analysis sensor 106 is disposed at the inlet of the flow restriction 104 (e.g., a Venturi) instead of at the outlet of the flow restriction 104.

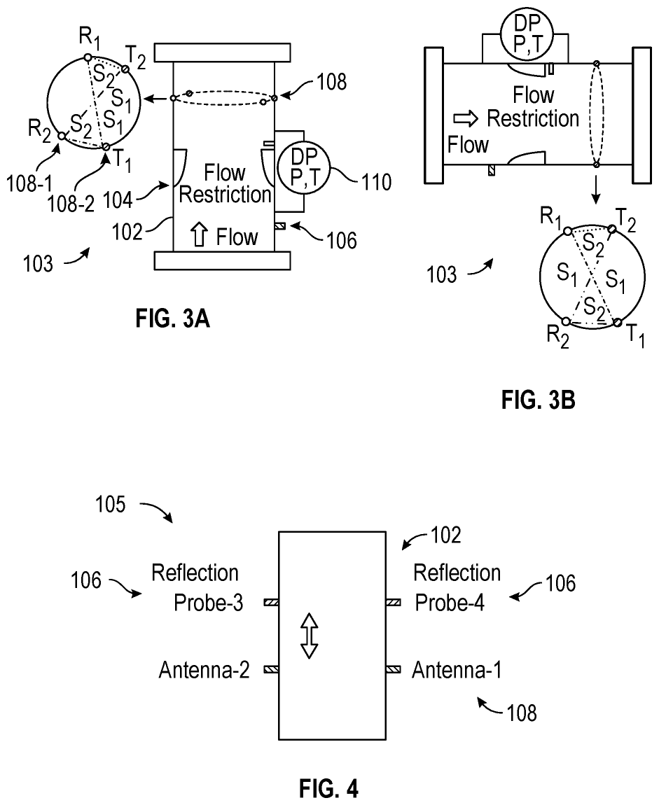

FIG. 3A and FIG. 3B illustrate an example of a system 103 showing another variant of the example embodiment of FIG. 1, which may be positioned in vertical configuration (FIG. 3A) or horizontal configuration (FIG. 3B). In the system 103, the flow restriction 104 is shaped via one or more inserts (e.g. a flow nozzle) fixed in the pipe to locally reduce the diameter of the conduit 102 (e.g., in a measurement spool pipe section). Such a flow restriction is particularly appropriate for geothermal wells having conduits of larger diameters. In the system 103 the differential pressure (DP) sensor 110 is installed between two locations, respectively upstream and downstream of the flow restriction 104. Such a differential pressure sensor may be for instance a multi-variable transmitter (MVT) to measure the DP across a flow restriction (e.g., and to measure steam flow P and T). The two pairs of antennas 108-1 and 108-2 are disposed in this embodiment downstream of the flow restriction 104, on a same pipe section. The antennas of each pair are disposed at opposite azimuthal locations of the pipe section with a near-spacing (near-wall) receiver and a far-spacing (cross-pipe) receiver, relative to a transmitter.

FIG. 4 illustrates another example variant 105 of a portion of the system 100 as per the example embodiment of FIG. 1. In this variant, the pair of antennas 108 (antenna-1 and antenna-2) and the water analysis sensor 106 (reflection probe-3 and reflection probe-4) are in the same pipe section of a conduit 102, which may be an appropriate section of the conduit 102 such as a Venturi throat, inlet or outlet if the flow restriction is a Venturi or upstream or downstream of a flow nozzle. The water analysis sensor 106 can be a microwave reflection probe or probes in the example embodiment of FIG. 4 where the water analysis sensor 106 is able to receive a signal from at least one of the transmitting antennas. Such a setup enables performance of transmission, resonance and reflection measurement of the antennas 108 by analyzing at least a portion of a signal transmitted by one of the antennas (e.g., antenna-1 or antenna-2) and received by the other of the antennas (e.g., antenna-2 or antenna-1) and the microwave reflection probe of the water analysis sensor 106. In such an example, the water analysis sensor 106 can utilize the microwave reflection probe as a receiving antenna that receives at least a portion of the transmitted signal. For example, a pair of antennas can include a transmitting antenna as a first antenna and a receiving antenna as a second antenna where the receiving antenna is part of the water analysis sensor 106 (e.g., the water analysis sensor can include an antenna suitable for receiving at least a portion of a transmitted signal).

As indicated, multiple probes may be utilized such as, for example, two opposing probes both labeled 106 and identified as reflection probe-3 and reflection probe-4 in FIG. 4. Utilization of more than one reflection probe (e.g., opposite another reflection probe) as in a 2-transmitter and 2-receiver configuration with the antennas 108 enables a compact setup to make an improved compensated measurement without integrating into the conduit a second pair of antennas. As an example, when the water analysis sensor 106 is a microwave reflection probe, a system may use such a microwave reflection probe as the water analysis sensor 106 and as the second (receiving) antenna of the pair of microwave antennas 108.

As explained, a system can include a processing system such as the processing system 114 of FIG. 1. Such a processing system can provide for fluid parameter determination using measurements such as sensor measurements acquired via one or more sensors of a system. As explained, a system can include a water analysis sensor and at least a pair of antennas where one of the antennas can be part of the water analysis sensor (e.g., utilized as a receiving antenna).

As an example, consider fluid parameter determination as may be suitable for a geothermal well, where there is one liquid phase (e.g., free water) and where a gas phase is a steam phase (e.g., water vapor); and, suitable for a hydrocarbon well, where fluid is a 3-phase fluid where liquid phases include an oil phase and a water phase.

As an example, a system can include a conduit; a water analysis sensor arranged on the conduit that measures at least a property of multiphase fluid in the conduit, where the multiphase fluid includes at least a gaseous phase and a liquid water phase; at least a pair of microwave antennas, where the pair of microwave antennas includes a first antenna that transmits a signal into the multiphase fluid in the conduit and a second antenna that receives at least a portion of the signal as transmitted into the multiphase fluid in the conduit; and a processing system that includes a processor, memory accessible to the processor, and processor-executable instructions stored in the memory and executable by the processor to cause the processing system to: determine at least a property of the liquid water phase based on output of the water analysis sensor, where the at least a property includes one or more of a water salinity and a water liquid ratio, determine one or more of a mixture permittivity and a mixture conductivity of the multiphase fluid based on output of the second antenna, and determine a gas hold-up of the multiphase fluid based at least in part on one or more of the mixture permittivity and the mixture conductivity. For example, consider one or more of the systems 100 of FIG. 1, 101 of FIG. 2, 103 of FIG. 3A and FIG. 3B and 105 of FIG. 4 as including such features.

As an example, a system can include equipment integrated locally and may include processing equipment integrated locally and/or remotely. For example, the processing system 114 of the example system 100 of FIG. 1 may be local and/or remote. As an example, the processing system 114 may be integrated into an assembly that includes the conduit 102 and/or that couples to the conduit 102.

As to the example conduit 102, it may be of one or more cross-sectional shapes (e.g., circular, oval, rectangular, etc.). As shown in the examples of FIG. 1 and FIG. 2, the conduit 102 can include end flanges such that the conduit 102 can be integrated into a flowline (e.g., a pipeline, etc.). As an example, the conduit 102 may be positioned proximate to a wellhead and/or another piece of equipment where the conduit 102 includes a lumen (e.g., a bore) that can receive multiphase fluid as produced by one or more wells. As an example, the conduit 102 may be configured to be inserted into a flowline, utilized, and then removed from the flowline (e.g., replaced with a pipe, etc.). As an example, the conduit 102 may be part of a well testing system for performing one or more well testing tasks. As an example, the conduit 102 may be part of a control system where one or more determinations made via one or more sensors coupled to the conduit 102 may be utilized to generate one or more control instructions that control one or more pieces of equipment in a flow network (e.g., a surface network that may be in fluid communication with one or more well, a processing facility, etc.).

### Example Case of Three-Phase Fluids.

In a first scenario, a pair of antennas can determine mixture permittivity (as well as quality factor indicative of the mixture conductivity) via resonance frequency (and frequency peak width) determination technique. This first scenario generally applies for a type of fluid that includes (high-GVF) oil-continuous (OC) or gas-continuous (GC) flow. Such a type of fluid is a fluid in which the phase that is considered continuous is oil or gas and the other phases are considered as inclusion in the continuous phase. As an example, such a scenario can be a default scenario. In a second scenario, as explained further below, generally for high-GVF water-continuous (WC) flow, resonance is absent and a pair of antennas can provide for determinations of transmission amplitude attenuation and phase shift at one or more frequencies.

In each of the example scenarios, the water/liquid ratio, also referred to as a water liquid ratio (WLR), may be determined from the parameters measured by a water analysis sensor. Various examples of models may enable WLR determination (see, e.g., the aforementioned published PCT application WO 2021/011477 A1 for a water analysis sensor being a microwave reflection probe determining near-wall fluid permittivity and conductivity).

### Determination of the GHU in First Scenario

In the first scenario, the (dominant TE₁₁ mode) resonance peak frequency (f_{c}) can be determined from a pair of antenna measurement and a bulk (cross-pipe, volume-averaged) mixture permittivity (ε_{mix,bulk}) can be determined by a processing system from the following equations:

$${f}_{c} = 0.293 {c}_{o} / ( 0.5 {D}_{T} \sqrt{{ε}_{mix , bulk}} ) , \qquad ( 1 )$$

$${ε}_{mix , bulk} = \sqrt{{f}_{c , empty} / {f}_{c}} \qquad ( 2 )$$

where D_{T} is the inner diameter of the metal pipe section in which the pair of antennas is installed (e.g., of the Venturi throat section in FIG. 1, for instance), cₒ is the velocity of electromagnetic wave in free space (close to that in empty pipe), f_{c,empty} is the empty-pipe (ε≈1) resonance peak frequency.

The determination of the fluid type of the flow (e.g., gas-continuous (GC), liquid continuous (LC), including oil-continuous (OC) or water-continuous (WC)) may be based on one or more techniques and on outcomes of a water analysis sensor and pair of antennas.

In an embodiment, for high-GVF flows (>95%), the absence of resonance from a pair of antennas measurement will show a WC flow. A water analysis sensor will generally measure a near-wall conductivity over a threshold for a WC flow containing brine water (e.g., more dissolved inorganic salt than typical seawater). When the received signal includes a resonance frequency, if the water analysis sensor measures a near-wall conductivity close to zero, the resonance frequency peak may be further examined. If the resonance frequency is close to that of the gaseous flow (at measured pressure P and temperature T) and the Q-factor is relatively high (i.e. a narrow/sharp peak), the flow is a GC flow. If resonance frequency close to that of the oil flow at (P, T) and the Q-factor is relatively low (e.g., a relatively broad peak), the flow is an OC flow.

The mixture permittivity may be calculated from a dielectric mixing model, for instance from a Ramu-Rao dielectric mixing model, complex refractive index method (CRIM) or any other appropriate mixing model.

As an illustration, for a liquid continuous (LC) gas-liquid flow, the gas holdup (GHU) may be derived from the bulk mixture permittivity ε_{mix,bulk} (e.g. determined from the resonance-peak frequency) and the WLR (e.g., determined by the water analysis sensor, e.g. a reflection probe), for example, from the CRIM-like dielectric-mixing equation:

$$GHU = {F}_{GL , lc}^{- 1} ( {ε}_{mix , bulk} , {ε}_{liq , lc} , {ε}_{gas} ) = \frac{\sqrt[m]{{ε}_{liq , lc}} - \sqrt[m]{{ε}_{mix , bulk}}}{\sqrt[m]{{ε}_{liq , lc}} - \sqrt[m]{{ε}_{gas}}} \qquad ( 3 )$$

where m is an empirical parameter (e.g. m=2). In the case of an oil continuous (OC) liquid, the homogeneous (oil-water) liquid permittivity (ε_{lig,lc}=ε_{lig,oc}) can be calculated from a suitable dielectric mixing model, such as simplified Ramu-Rao dielectric mixing models using known oil permittivity and calculated WLR as inputs:

$${ε}_{liq , oc} = {F}_{L , oc} ( {ε}_{oil} , WLR ) = {ε}_{oil} \frac{1 + 2 WLR}{1 - WLR} \qquad ( 4 )$$

For a (fresh) water continuous (WC) liquid, the homogeneous (oil-water) liquid permittivity (ε_{liq,lc}=ε_{lig,wc}) can be calculated from a suitable dielectric mixing model such as the simplified Ramu-Rao using known or calculated parameters of water permittivity and WLR (e.g. from a water analysis sensor), viz.

$${ε}_{liq , wc} = {F}_{L , wc} ( {ε}_{water} , WLR ) = {ε}_{water} \frac{2 WLR}{3 - WLR} \qquad ( 5 )$$

A gas continuous (GC) flow (with water fraction α_{water} and oil fraction αₒᵢₗ) may be treated based on a simplistic CRIM-like dielectric mixing model, viz.

$${ε}_{mix , bulk} = {[ {α}_{water} \sqrt[m]{{ε}_{water}} + {α}_{oil} \sqrt[m]{{ε}_{oil}} + ( 1 - {α}_{water} - {α}_{oil} ) \sqrt[m]{{ε}_{gas}} ]}^{m} \qquad ( 6 a )$$

Since

$$WLR = \frac{{α}_{water}}{{α}_{water} + {α}_{oil}} = \frac{{α}_{water}}{{α}_{liquid}} = \frac{{α}_{water}}{1 - GHU} ,$$

the following can then be derived:

$${ε}_{mix , bulk} = {[ WLR ( 1 - GHU ) \sqrt[m]{{ε}_{water}} + ( 1 - WLR ) ( 1 - GHU ) \sqrt[m]{{ε}_{oil}} + GHU \sqrt[m]{{ε}_{gas}} ]}^{m} \qquad ( 6 b )$$

noting that

$${ε}_{liq , gc} = {[ WLR \sqrt[m]{{ε}_{water}} + ( 1 - WLR ) \sqrt[m]{{ε}_{oil}} ]}^{m} \qquad ( 6 c )$$

It is possible to derive an approximation of GHU from the foregoing example Equations (6b-6c), given the ε_{mix,bulk} (derived from the measured resonance frequency) and ε_{liq,gc} (derived from the WLR by using the water analysis sensor, e.g. a near-wall measurement reflection probe):

$$GHU = {F}_{GL , gc}^{- 1} ( {ε}_{mix , bulk} , {ε}_{liq , gc} , {ε}_{gas} ) = \frac{\sqrt[m]{{ε}_{liq , gc}} - \sqrt[m]{{ε}_{mix , bulk}}}{\sqrt[m]{{ε}_{liq , gc}} - \sqrt[m]{{ε}_{gas}}} gas continuous \qquad ( 7 )$$

where ε_{liq,gc} may be calculated from Equation (6c) that assumes no oil- or water-continuous regime. Note Equation (7) is somewhat similar to Equation (3).

Gas-continuous (mist) wet-gas flow may be more accurately characterized from the additional electromagnetic (EM) modeling of mist-flow dielectric mixing model that is described below. Determining accurately the GHU in such mist-flow is a challenge that has not been previously resolved and is detailed in the below section.

### Particular Example Case of First Scenario: GHU Determination in Gas-Continuous (Mist) Wet-Gas Flow

As previously indicated, in the particular case of high-GVF wet gas flows (mist flow), previous models have not been able to determine easily the parameters of interest and the GHU determination may be enhanced using a method as will be described below.

Indeed, two-phase dielectric mixing models, such as the Ramu-Rao, Bruggeman, Maxwell-Garnett and others dielectric mixing models, are available for a homogenous mixture of oil and water, or for a homogenous mixture of liquid and gas with spherical inclusions.

However, in the mist flow regime where the GVF is typically above 95% or even 98%, there may be a lack of liquid for oil and water to be mixed, causing them to be discrete oil and water droplets. Therefore, the existing mixing formulas for a mixture of two fluids with spherical inclusions can be adapted for use in the mist flow regime so that an accurate GHU can be retrieved. An incorrect usage of the mixing formulas can cause the effective permittivity to deviate due to the high permittivity contrast between water (ε≈80) and hydrocarbon (ε≈1 to 2.1). The host and inclusions are to be selected appropriately as well, noting that knowledge of “phase inversion” in mist flow can be lacking.

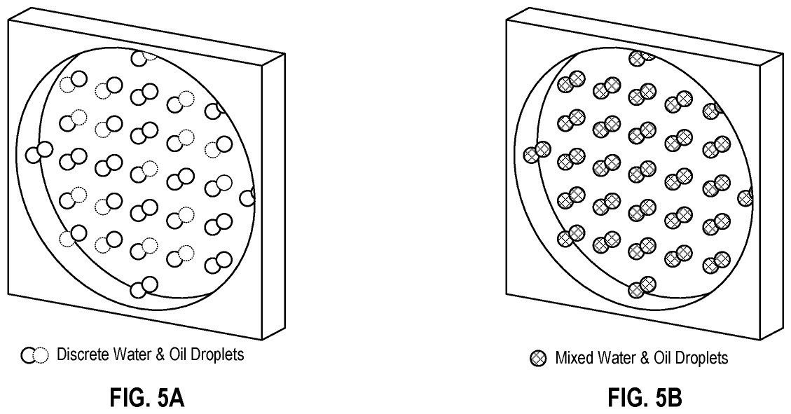

To further illustrate this aspect, FIG. 5A shows a cross-section of a wet-gas flow pipe in a mist-flow regime, with discrete oil and water droplets, configuration which is not taken into account by the conventional dielectric mixing model, and FIG. 5B shows a pipe with a cross section of a pipe with homogeneously mixed oil and water droplets, which is the configuration taken into account by the conventional dielectric mixing model.

The effective permittivity (dielectric constant) ε_{mix,bulk} as determined via Equation (2) is reproduced below. The quality factor Q_{d} of a fluid mixture inside the metal pipe (waveguide) is related to the measured resonance peak frequency f_{C} and the (3-dB) peak width Δf_{c}, and is also expressed below:

$${ε}_{mix , bulk} = \sqrt{{f}_{c , empty} / {f}_{c}} \qquad ( 2 )$$

$${Q}_{d} = \frac{{f}_{c}}{Δ {f}_{c}} \qquad ( 2 b )$$

The conventional methods (see, e.g., Equations (6a-6b)) may be used to calculate theoretically the three-phase mist-flow mixture permittivity in the high-GVF gas continuous mist flow, without capturing the liquid being in an oil continuous (OC) regime, water continuous (WC) regime or in a “phase-inversion” transition regime.

For some of the existing dielectric mixing formulas that can capture the WC or OC liquid regime, two physically feasible theoretical calculations of the three-phase mist-flow mixture permittivity (as input to an appropriate 3-dimensional EM simulation) can be made, with the following:
a) The liquid mixture permittivity is calculated first, with oil as the continuous phase (OC) and water as the inclusions. The liquid mixture is then mixed with the gas phase, with liquid as the inclusions.
b) The liquid mixture permittivity is calculated first, with water as the continuous phase (WC) and oil as the inclusions. The liquid mixture is then mixed with the gas phase, with liquid as the inclusions.

The simulated effective permittivity of discrete water-oil droplets follows the value calculated by Ramu-Rao (or equivalent Maxwell-Garnett) mixing formula for OC mixed liquid for WLR up to a first threshold, of approximately 0.7, then gradually jumps to the value calculated by Ramu-Rao formula for WC mixed liquid for WLR starting at a second threshold, for instance 0.8.

Therefore, a 3D electromagnetic (EM) simulation (and some experimental measurements) provides that existing appropriate two-phase dielectric mixing laws, such as the Ramu-Rao or Maxwell-Garnett formulas can be extended for an accurate characterization of the effective permittivity of three-phase mist flows over a full range of WLR even at high GVF or GHU.

For determining the GHU in a mist flow, the processing system 114 can be therefore configured to first determine WLR. The WLR may be determined using a water analysis sensor, such as a reflection probe. Using a thresholding process, the processing system 114 may determine if the mist-flow mixture is in a water continuous (WC) region or in an oil continuous (OC) region. If WLR is determined by a microwave reflection probe, for instance, the thresholding process may be as follows: if WLR is above a (second) threshold, such as 80%, the liquid in the mist-flow mixture is in the water continuous region (a WC mist-flow mixing model is used by the processing system 114 to calculate GHU, given the resonance-frequency determined mist-flow effective permittivity). If WLR is below 70%, the liquid in the mist-flow mixture is in the oil continuous region (an OC mist-flow mixing model is used by the processing system 114 to calculate GHU, given the mist-flow effective permittivity). If WLR is between 70% and 80%, the liquid in the mist-flow mixture is in the phase inversion region (a hybrid of WC and OC mist-flow mixing models may be used by the processing system 114 to calculate GHU, given the mist-flow effective permittivity). However, depending on the fluid properties and phase fractions at the prevailing process pressure and temperature, the thresholds may change and/or the phase inversion region may disappear. The quality factor obtained from the pair of microwave antennas may be used in addition by the processing system 114 to confirm the extent of WC, OC or phase-inversion regions, especially in cases where the GVF is very high (ca. >99.5 or 99.9%) where some of the water analysis sensors in WLR determination may not be as accurate as expected.

In another embodiment, the processing system 114 may use differences in measured transmission amplitudes (and/or in the measured phase-shift data) at a resonance frequency and close to a resonance frequency, to determine WLR and GHU. Amplitude attenuation and phase shift determination are indeed also properties that may be measured from the signal received by the second antenna. The processing system 114 may use an appropriate regression, interpolation or machine-learning method to obtain the WLR and GHU, from the data measured by the water analysis sensor, and the measured resonance frequency, quality factor, differences in the measured transmission amplitudes (e.g., amplitude attenuation) or phases (e.g., phase shift) (in particular at the resonance frequency and close to the resonance frequency). Such method may be implemented in particular for flows having a very high GVF.

As an example, such a method may be implemented in particular for flows having a very high GVFs (e.g. >99.5%) that may not provide reliable WLR or GHU determination via water analysis sensor and/or for gas continuous (GC) mist flow that includes a phase-inversion region that is different from a liquid-continuous flow, for which the Equations (3)-(5) above may not be accurate.

As an example, the processing system 114 may determine liquid mixture permittivity using the liquid (oil and water) Ramu-Rao (or Maxwell-Garnett) mixing model, with the WLR as input. For the water continuous (WC) region, the host is water and inclusions are oil droplets. For the oil continuous (OC) region, the host is oil and inclusions are water droplets. For the phase inversion region, both water continuous (WC) region and oil continuous (OC) region may be calculated and the liquid mixture permittivity may be calculated as a combination (linear average or weighted average) of the liquid mixture permittivity returned by both models.

As an example, the processing system 114 can then be configured to determine GHU using the liquid-in-gas Ramu-Rao (or Maxwell-Garnett) mixing models, with the resonance-frequency determined gas-liquid mixture/effective permittivity and WLR as input parameters, as well as the liquid mixture permittivity determined as explained above.

### Example Second Scenario:

For salty-water water-continuous (wet-gas) flows, bulk mixture permittivity (and mixture conductivity) may be determined from the measured transmission attenuation (AT) and phase-shift (PS) at selected radio frequencies, such as that described in a US patent U.S. Pat. No. 7,908,930 B2, granted 22 Mar. 2011, which is herein incorporated by reference. As explained, in this case, there is generally no resonance frequency.

Denoting L as the effective spacing between a transmission antenna and a receiving antenna, the transmission amplitude-attenuation AT (in dB) and phase-shift PS (in radian), relative to the empty-pipe values, may be related to the attenuation constant αₘ (in neper/m) and the phase constant βₘ (in radian/m) as:

$${α}_{m} = AT / ( 8.686 L ) \qquad ( 8 a )$$

$${β}_{m} = PS / L \qquad ( 8 b )$$

The mixture permittivity εₘᵢₓ and the mixture conductivity σₘᵢₓ can be expressed as:

$${ε}_{mix} = {( {c}_{o} / ω )}^{2} ( {k}_{c}^{2} - {α}_{m}^{2} + {β}_{m}^{2} ) \qquad ( 9 a )$$

$${σ}_{mix} = 2 {α}_{m} {β}_{m} / ( ωμ ) \qquad ( 9 b )$$

where k_{c} is the cutoff wavenumber of the TE₁₁ dominant mode (k_{c}=1.84/a, with a being the pipe section inner radius), ω the angular frequency, μ the free-space magnetic permeability.

Once the bulk mixture permittivity εₘᵢₓ has been determined, the bulk mixture permittivity may be used as an input to determine the GHU using a dielectric mixing model as in Equations (3) and (5). The bulk mixture conductivity may also additionally or alternatively be used to determine the GHU, by using the mixing model below:

$$GHU = {F}_{GL , wc}^{- 1} ( {σ}_{mix} , {σ}_{liquid} ) = 1 - {( {σ}_{mix} / {σ}_{liquid} )}^{m} \qquad ( 10 )$$

where the water-continuous liquid conductivity (σ_{liq}) is calculated from a suitable dielectric mixing model such as the simplified Ramu-Rao using known and/or calculated parameters such as water conductivity σ_{water} and WLR from the water analysis sensor, viz.

$${σ}_{liquid} = {F}_{L , wc} ( {σ}_{water} , WLR ) = {σ}_{water} \frac{2 WLR}{3 - WLR} \qquad ( 5 b )$$

noting that Equation (5b) is a counterpart of the Equation (5).

### Summary of GHU Determination in Three-Phase High-GVF Flow

Below, a summary is presented for examples of fluid models used for GHU determination in a 3-phase high-GVF flow:
a) for high-GVF oil continuous (OC), (fresh) water continuous (WC) or gas-continuous (GC) mist flow (with WLR as input obtained from a water analysis sensor), GHU is determined from resonance-frequency derived (bulk) mixture permittivity:
   i) if fluid type is OC, select OC gas-liquid dielectric-mixing model(s), or
   ii) if fluid type is WC, select WC gas-liquid dielectric-mixing model(s)
   iii) if fluid type is GC, select GC gas-liquid dielectric-mixing model(s), as described in particular embodiment
b) for high-GVF water continuous (WC) flow (resonance generally absent), GHU is determined from transmission amplitude attenuation/phase shift derived mixture permittivity or mixture conductivity, with WLR, water permittivity and water conductivity from the water analysis sensor as inputs
   i) select WC gas-liquid dielectric-mixing model(s)

### Determination of the Flow Rate of Each Phase of the Three-Phase Fluid

Combining differential pressure measured across the flow restriction with the WLR and GHU determined by the water analysis sensor and the pair(s) of transmission antennas, the flow rates of the oil, water and gas can be determined from a flow rate determination model, as will explained below. The flow rate determination can involve use of an appropriate flow (slip) model.

As an example, the processing system 114 may determine oil-water liquid density μ_{liquid} and gas-liquid flow mixture density μₘᵢₓ from WLR and GHU as well as known densities of each fluid as follows:

$${ρ}_{liquid} = {ρ}_{water} WLR + {ρ}_{oil} ( 1 - WLR ) \qquad ( 11 )$$

$${ρ}_{mix , T} = {ρ}_{liquid} ( 1 - GHU ) + {ρ}_{gas} ( GHU ) \qquad ( 11 b )$$

The processing system 114 may then calculate the gas-liquid flow total mass flowrate from DP measurement as:

$${Q}_{total} = \sqrt{2 / ( 1 - {β}^{4} )} {C}_{d} ( R e ) {A}_{T} \sqrt{{ρ}_{mix , T} Δ P} \qquad ( 12 )$$

where C_{d} is the discharge coefficient which is Reynolds-number (Re) dependent, AT the venturi throat area (e.g., in the case of a flow restriction being a Venturi).

As indicated, a gas-liquid flow slip model (e.g., at the microwave transmission measurement section) may be used to determine the gas volume fraction (GVF) as a function of the GHU, differential pressure and density and viscosity of gas and liquid (with u being the viscosity). An appropriate slip model may be used to determine GVF. The homogenous mixture density ρ_{H} may be directly derived from the GVF determination.

$$GVF = \frac{{q}_{gas}}{{q}_{total}} = func ( GHU , \frac{{ρ}_{gas} ( p , T )}{{ρ}_{liquid} ( p , T )} , \frac{{μ}_{gas} ( p , T )}{{μ}_{liquid} ( p , T )} ; Δ P ) \qquad ( 13 )$$

$${ρ}_{H} = {ρ}_{liquid} ( 1 - GVF ) + {ρ}_{gas} GVF \qquad ( 14 )$$

The gas volumetric flowrate (q_{gas}), oil volumetric flowrate (qₒᵢₗ) and water volumetric flowrate (q_{water}) can be calculated from the total volumetric flowrate (qₜₒₜₐₗ) using the GVF and WLR as per formula such as, for example:

$${q}_{total} = {Q}_{total} / {ρ}_{H} \qquad ( 15 a )$$

$${q}_{gas} = {q}_{total} GVF \qquad ( 15 b )$$

$${q}_{oil} = {q}_{total} ( 1 - GVF ) ( 1 - WLR ) \qquad ( 15 c )$$

$${q}_{water} = {q}_{total} ( 1 - GVF ) WLR \qquad ( 15 d )$$

Therefore the processing system 114 can be configured to determine the flow rate of each of the phase of the 3-phase fluid based on the input of the water analysis, pair(s) of transmission antennas and differential pressure sensors and a priori models and various equations.

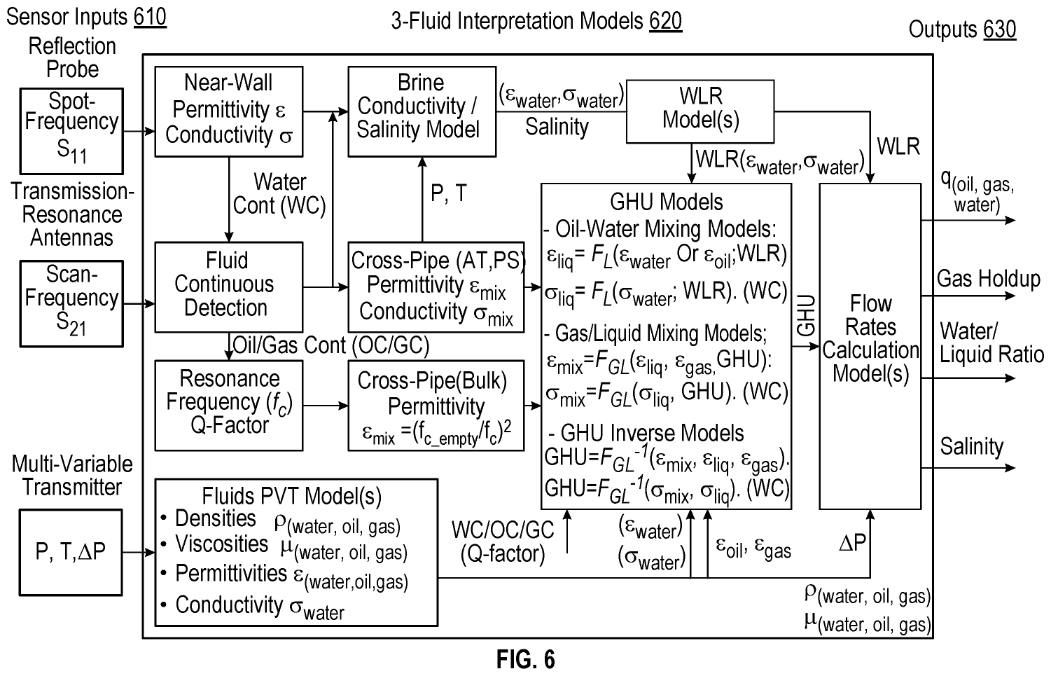

FIG. 6 shows a block diagram that includes examples of sensor input 610, examples of three-phase fluid interpretation models 620 and example outputs 630. The block diagram of FIG. 6 provides examples of various parameters that can be determined from sensor measurement in a three-phase fluid case. In this embodiment, a water analysis sensor is a microwave reflection probe and a differential pressure sensor is a multi-variable transmitter. A water salinity may therefore be determined from the water analysis sensor in additional to the WLR. The WLR and water salinity determinations are performed using known models such as the model described in the aforementioned published PCT application WO 2021/011477 A1. The multi-variable transmitter measures pressure (P) and temperature (T) on top of differential pressure that enables to derive, via the use of fluids pressure-volume-temperature (PVT) model(s), more precisely the values of density ρ, viscosity μ, dielectric permittivity ε and electrical conductivity σ for the different fluids as shown on FIG. 6.

### Example Case of a Geothermal Well.

In this case, as mentioned above, the multiphase fluid flow is a steam flow including a first (liquid) phase of free water and a second (gas) phase of water vapor, with both low and high steam quality.

As for a 3-phase fluid, a system can include a flow restriction, a water analysis sensor, at least one pair of microwave antennas and a differential pressure sensor.

In a first scenario, the pair of antennas can determine the mixture permittivity as well as the quality factor (indicative of the mixture conductivity) via resonance frequency determination technique. This first scenario generally applies for a type of fluid includes (high-GVF) vapor-continuous flow. Such a type of fluid is a fluid in which the phase that is considered continuous is vapor and the liquid water phase is considered as inclusion in the continuous phase. This scenario, as explained, may be the default scenario. In a second scenario, generally for high-GVF water-continuous (WC) flow when the salinity of the water is high, as explained, resonance can be absent and a pair of antennas can determine amplitude attenuation and phase shift at one or more frequencies.

In each of the scenario, the water salinity may be determined from the parameters measured by the water analysis sensor. Known models may enable water salinity determination. For instance, such models are described in a US patent (U.S. Pat. No. 6,831,470 B2, granted 14 Dec. 2004) and in the aforementioned PCT application (WO 2021/011477 A1), both of which are incorporated by reference herein in their entirety, including for a water analysis sensor being a microwave reflection probe for determining near-wall fluid permittivity and conductivity. In some cases, water salinity may also be derived from water-continuous flow mixture permittivity and mixture conductivity determined from the AT and PS data measured by pair of transmission antennas (as described in U.S. Pat. No. 6,831,470 B2).

### Determination of the GHU in First Scenario

As explained in relationship with the 3-phase fluid, in a first scenario, the (dominant TE₁₁ mode) resonance peak frequency (f_{c}) is determined from the pair of antenna measurement and the bulk (cross-pipe, volume-averaged) mixture permittivity (ε_{mix,bulk}), is determined by the processing system from the Equations (1) and (2) mentioned above.

For a high steam-quality vapor- or steam continuous (SC) flow identified for instance by the low values in the near-wall mixture permittivity measured by the reflection probe, the gas holdup GHU (that relates to the water holdup water via the following relationship: GHU=1−α_{water}) at the measurement section may be derived from the resonance-mode determined bulk mixture permittivity ε_{mix,sc} via a suitable dielectric mixing model, such as simplified Ramu-Rao mixing model below:

$$GHU = 1 - ( \frac{{ε}_{mix , sc}}{{ε}_{steam}} - 1 ) / ( \frac{{ε}_{mix , sc}}{{ε}_{steam}} + 2 ) \qquad ( 16 )$$

Steam/vapor permittivity εₛₜₑₐₘ (and free water permittivity ε_{water}) are well-known. In an embodiment, they may be determined by a PVT-like model, given the pressure (p) and temperature (T) that may be obtained via a measurement, such as a multi-variable transmitter.

For low steam-quality (free) water continuous (WC) flow, the transmission-mode mixture permittivity (ε_{mix,wc}) can be related to the GHU via an appropriate dielectric mixing model, such as that based on the complex refractive index method (CRIM):

$$GHU = 1 - \frac{{( {ε}_{mix , wc} / {ε}_{water} )}^{m} - {( {ε}_{steam} / {ε}_{water} )}^{m}}{1 - {( {ε}_{steam} / {ε}_{water} )}^{m}} \qquad ( 17 )$$

The mixture conductivity (σ_{mix,wc}) may additionally or alternatively be used to calculate GHU from the following CRIM-like mixing model:

$$GHU = 1 - {( {σ}_{mix , wc} / {σ}_{water} )}^{m} \qquad ( 18 )$$

Brine water conductivity σ_{water} in Equation (18) and the water permittivity ε_{water} in Equation (17) may be determined based on the water salinity derived by the water analysis sensor, for example, a reflection probe.

### Example Second Scenario

The mixture permittivity (ε_{mix,wc}) and mixture conductivity (σ_{mix,wc}) can be related to the measured transmission (AT, PS) data (from one or more receivers, and at one or more transmission frequencies), as described in Equations (8a), (8b), (9a) and (9b). The GHU is determined from Equation (17) or (18).

The water conductivity σ_{water} (and salinity s) and water permittivity ε_{water} can be determined from this ratio σ_{mix,wc}/ε_{mix,wc} and from the measured fluid temperature (7) and pressure (φ, by using an extended (NaCl) brine dielectric model. Hence, consider the following equation:

$$( {σ}_{water} , {ε}_{water} ; s ) = {f}_{1} ( \frac{{σ}_{mix , wc}}{{ε}_{mix , wc}} , T , p ) \qquad ( 19 )$$

### Summary of GHU Determination

In a geothermal fluid, with oil being absent, WLR is constant (e.g., WLR=constant=1). Below is a summary of an example of fluid models that can be used for GHU determination in such a geothermal fluid:
(a) salinity (water-conductivity) can be determined by ae water analysis sensor,
(b) for GHU determination:
   (i) in case of free-water [liquid] absence or being entrained as droplets (vapor/gas continuous, GC), GHU can be determined by microwave resonance data using a gas continuous (GC) dielectric mixing model. Water salinity determined by the reflection probe may be used as an input but generally has a negligible effect so GHU in this case may also be calculated without water salinity input,
   (ii) in case of free-water [liquid] presence as full or partial liquid wall layer (water continuous, WC), in particular, when water salinity is high, GHU can be determined by microwave transmission data using a water continuous (WC) dielectric model. The water salinity can be determined by a reflection probe as used as an input for determining the GHU.

### Determination of the Flow Rate of Each Phase of the Geothermal Fluid.

Combining differential pressure measured across a flow restriction with water salinity, water permittivity and water conductivity determined by a water analysis sensor and GHU determined by a pair of antennas, flow rates of water and steam (and the steam quality and enthalpy) can be determined from a flow rate determination model, as explained below. As mentioned, flow rate determination can involve use of an appropriate flow (slip) model.

As an example, a processing system 114 may first determine steam-water flow mixture density using such formula:

$${ρ}_{mix} = {ρ}_{water} ( 1 - GHU ) + {ρ}_{steam} GHU \qquad ( 20 )$$

In Equation (20), the water density ρ_{water} and steam density ρₛₜₑₐₘ are known and may be determined at current pressure and temperature via appropriate PVT model. Water salinity may be also taken into account in the water density determination.

Then, the processing system 114 can determine steam-flow total mass flowrate using for instance the Equation (21), in case the flow restriction is a Venturi:

$${Q}_{total} = \sqrt{2 / ( 1 - {β}^{4} )} {C}_{d} ( R e ) {A}_{T} \sqrt{{ρ}_{mix , T} Δ P} \qquad ( 21 )$$

where C_{d} is the discharge coefficient which is Reynolds-number (Re) dependent, AT the venturi throat area (in the case of a flow restriction being a Venturi). One or more other equations may be used in case of another setup or configuration being used.

As an example, the processing system 114 may then establish the gas(vapor)-liquid(water) flow slip model (e.g., at the microwave transmission-resonance measurement section) that can be used to determine the vapor or gas volume fraction (GVF) and the homogenous mixture density ρ_{H} (here μ being viscosity):

$$GVF = \frac{{q}_{steam}}{{q}_{total}} = func ( GHU , \frac{{ρ}_{steam} ( p , T )}{{ρ}_{water} ( p , T )} , \frac{{μ}_{steam} ( p , T )}{{μ}_{water} ( p , T )} ; Δ P ) \qquad ( 22 )$$

$${ρ}_{H} = {ρ}_{water} ( 1 - GVF ) + {ρ}_{steam} GVF \qquad ( 23 )$$

Using the relationships above, the processing system 114 can be configured to obtain the total volumetric flowrate (qₜₒₜₐₗ), steam volumetric flowrate (qₛₜₑₐₘ) and the water volumetric flowrate (q_{water}), and the steam quality (x), from below formulas:

$${q}_{total} = {Q}_{total} / {ρ}_{H} \qquad ( 24 a )$$

$${q}_{steam} = {q}_{total} GVF \qquad ( 24 b )$$

$${q}_{water} = {q}_{total} ( 1 - GVF ) \qquad ( 24 c )$$

$$x = \frac{{Q}_{steam}}{{Q}_{total}} = \frac{{q}_{steam} \times {ρ}_{steam}}{{q}_{total} \times {ρ}_{H}} = GVF \frac{{ρ}_{steam}}{{ρ}_{H}} \qquad ( 24 d )$$

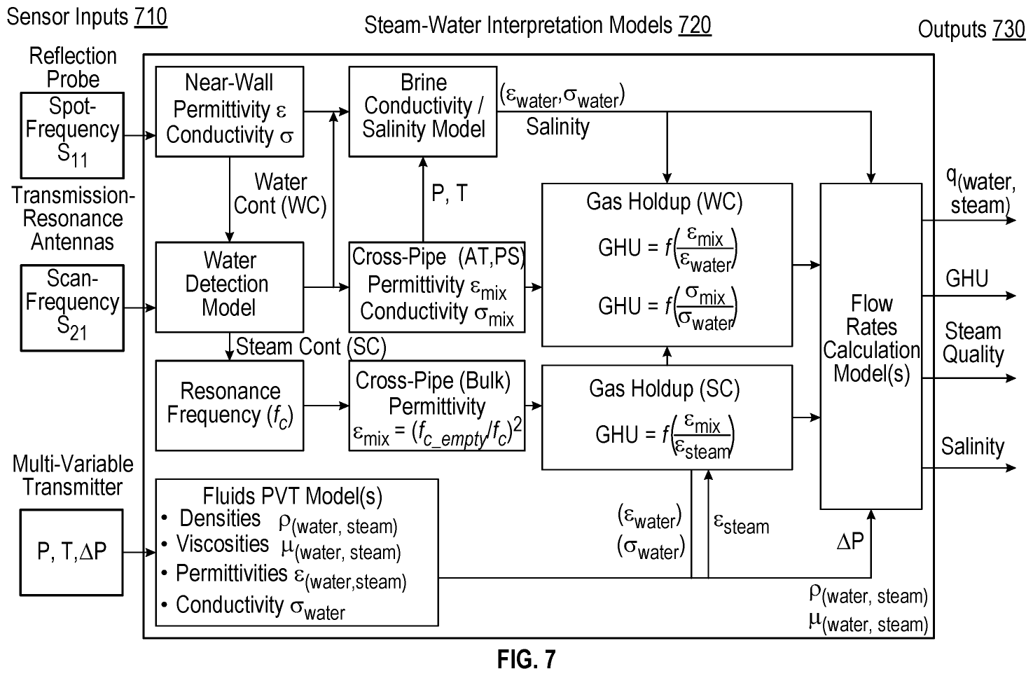

FIG. 7 shows a block diagram that includes examples of sensor input 710, examples of steam-water interpretation models 720 and example outputs 730. The block diagram of FIG. 7 provides examples of various parameters that may be determined from sensor measurement in a geothermal fluid case, according to an embodiment of the disclosure. In this embodiment, a water analysis sensor can be a microwave reflection probe and a differential pressure sensor can be a multi-variable transmitter. A multi-variable transmitter can measure pressure and temperature on top of differential pressure that enables derivation of more precise values of density ρ, viscosity μ, dielectric permittivity ε and electrical conductivity σ for the different fluids as shown on FIG. 7.

Various different embodiments of the systems and methods disclosed herein can enable determination of GHU of multiphase fluid using a water analysis sensor as well as a pair of microwave antennas. When associated to a flow restriction and a differential pressure measurement, flow rates of each phase of the fluid may also be derived. In various examples, one or more systems and/or methods may be applicable even for high-GVF flow measurements in which the flow rates determination tends to be quite challenging. As explained above, various examples systems and/or methods can be used in one or more of several types of flows such as 2- and 3-phase fluids and in numerous applications such as high-GVF wet gas flow, topside, subsea or geothermal applications.

FIG. 8 shows an example of a method 800 that includes a reception block 810 for receiving flowing multiphase fluid in a conduit, an acquisition block 820 for acquiring measurements with respective sensors relative to one or more properties of the multiphase fluid, where the sensors can include at least a water analysis sensor and a pair of microwave antennas, where the pair of microwave antennas includes a first antenna for transmitting a signal and a second antenna for receiving at least a portion of the signal, a determination block 830 for determining at least a property of a liquid water phase of the multiphase fluid based on an acquired water analysis sensor measurement, where the at least a property includes one or more of a water salinity and a water liquid ratio; a determination block 840 for determining one or more of a mixture permittivity and a mixture conductivity of the multiphase fluid based on the at least a portion of the signal received by the second antenna of the pair; a determination block 850 for determining a gas hold-up of the multiphase fluid based at least on the mixture permittivity or the mixture conductivity.

The method 800 is shown in FIG. 8 as including various computer-readable storage medium (CRM) blocks 811, 821, 831, 841, and 851 that can include processor-executable instructions that can instruct a computing system, which can be a control system, to perform one or more of the actions described with respect to the method 800. As an example, the processing system 114 can include instructions associated with one or more of the CRM blocks 811, 821, 831, 841 and 851 of the method 800 of FIG. 8 such that the processing system 114 can execute such instructions to cause the processing system 114 to perform at least part of the method 800. In such an example, instructions can include control instructions that can control operation of one or more pieces of equipment.

As an example, a system can include a conduit; a water analysis sensor arranged on the conduit that measures at least a property of multiphase fluid in the conduit, where the multiphase fluid includes at least a gaseous phase and a liquid water phase; at least a pair of microwave antennas, where the pair of microwave antennas includes a first antenna that transmits a signal into the multiphase fluid in the conduit and a second antenna that receives at least a portion of the signal as transmitted into the multiphase fluid in the conduit; and a processing system that includes a processor, memory accessible to the processor, and processor-executable instructions stored in the memory and executable by the processor to cause the processing system to: determine at least a property of the liquid water phase based on output of the water analysis sensor, where the at least a property includes one or more of a water salinity and a water liquid ratio, determine one or more of a mixture permittivity and a mixture conductivity of the multiphase fluid based on output of the second antenna, and determine a gas hold-up of the multiphase fluid based at least in part on one or more of the mixture permittivity and the mixture conductivity. In such an example, the gas hold-up can be determined based at least in part on the at least a property of the water liquid phase and one or more of the mixture permittivity and the mixture conductivity.

As an example, a system can include a water analysis sensor that can include a microwave reflection probe that transmits a microwave signal to multiphase fluid in a near-wall portion of a conduit and that receives at least a portion of the microwave signal as a reflected portion by the multiphase fluid and such a system can include a processing system that includes processor-executable instructions stored in the memory and executable by the processor to cause the processing system to determine one or more of a near-wall mixture conductivity and a near-wall mixture permittivity of a near-wall portion of the multiphase fluid, where at least a property of a liquid water phase is determined based at least in part on one or more of the near-wall mixture conductivity and the near-wall mixture permittivity.

As an example, a system can include a flow restriction in a conduit and a differential pressure sensor that measures a pressure difference between a first location upstream of the flow restriction and a second location in the flow restriction or downstream of the flow restriction. In such an example, the flow restriction can be a Venturi. As an example, a pair of microwave antennas can be positioned in a throat of a Venturi. As an example, a differential pressure sensor can be a multi-variable transmitter that acquires at least one measurement of one or more of a temperature and a pressure at one of first and second locations.

As an example, a system can include a processing system that includes processor-executable instructions stored in the memory and executable by the processor to cause the processing system to determine a flow rate of at least one phase of multiphase fluid based at least in part on at least a property of a liquid water phase, a gas hold-up and a differential pressure measurement.

As an example, a mixture permittivity of multiphase fluid can be determined by a processing system based on output of a second antenna of a pair of antennas where the mixture permittivity can be based at least in part on one or more of a determination of a resonance frequency of at least a portion of a signal received by the second antenna and a determination of a phase shift and/or amplitude attenuation at one or more frequencies of at least a portion of a signal received by the second antenna.

As an example, at least one pair of antennas can include two pairs of antennas. For example, a pair of antennas can include four antennas arranged as two pairs.

As an example, a water analysis sensor can include a microwave reflection probe that transmits a microwave signal to multiphase fluid where, for example, the microwave reflection probe can operate as a second antenna of a pair of antennas (e.g., to receive at least a portion of a signal transmitted into the multiphase fluid, which may be a transmitted and/or a reflected portion, etc.).

As an example, multiphase fluid can be a three-phase fluid that includes a liquid water phase, a gaseous phase and a liquid oil phase.

As an example, a processing system can include processor-executable instructions stored in memory and executable by a processor to cause the processing system to determine a type of flow of multiphase fluid as being an oil continuous flow, a water continuous flow, or a gas continuous mist flow. In such an example, the processing system can include processor-executable instructions stored in the memory and executable by the processor to cause the processing system to determine at least a water liquid ratio based at least in part on output of a water analysis sensor and to determine a gas hold-up based at least in part on the water liquid ratio, a mixture permittivity and/or a mixture conductivity and a dielectric mixing model, where the dielectric mixing model can be selected based on the type of flow. In such an example, for determining the gas hold-up when the type of flow is determined to be a mist flow, the processing system can include processor-executable instructions stored in the memory and executable by the processor to cause the processing system to: determine a liquid mixture permittivity based on one or more of an oil continuous dielectric mixing model and a water continuous dielectric mixing model using the water liquid ratio; and determine a gas hold-up based on a gas continuous dielectric mixing model using the liquid mixture permittivity and a mist flow mixture permittivity.

As an example, a dielectric mixing model can be a Ramu-Rao or Maxwell-Garnett dielectric mixing model.

As an example, a system can include a processing system that includes processor-executable instructions stored in memory and executable by a processor to cause the processing system to: determine a liquid mixture permittivity based on a water continuous dielectric mixing model for a water liquid ratio over a first threshold; determine the liquid mixture permittivity based on an oil continuous dielectric mixing model for a water liquid ratio under a second threshold; and determine the liquid mixture permittivity based on a combination of a first liquid mixture permittivity determined based on an oil continuous dielectric mixing model and a second liquid mixture permittivity based on a water continuous dielectric mixing model when the water liquid ratio is between the first and second threshold.

As an example, multiphase fluid can be a two-phase fluid that includes a water liquid phase and a steam phase as a gaseous phase where a processing system can include processor-executable instructions stored in memory and executable by a processor to cause the processing system to determine a gas hold-up of the multiphase fluid based at least in part on a mixture permittivity or a mixture conductivity and on a water salinity. In such an example, the processing system can include processor-executable instructions stored in the memory and executable by the processor to cause the processing system to determine the gas hold-up of the multiphase fluid based at least in part on the mixture permittivity or the mixture conductivity and on one or more of a water permittivity and a water conductivity derived from the water salinity.

As an example, a method can include receiving flowing multiphase fluid in a conduit; acquiring measurements with respective sensors relative to one or more properties of the multiphase fluid, where the sensors include at least a water analysis sensor and a pair of microwave antennas, where the pair of microwave antennas can include a first antenna for transmitting a signal and a second antenna for receiving at least a portion of the signal; determining at least a property of a liquid water phase of the multiphase fluid based on an acquired water analysis sensor measurement, where the at least a property includes one or more of a water salinity and a water liquid ratio; determining one or more of a mixture permittivity and a mixture conductivity of the multiphase fluid based on the at least a portion of the signal received by the second antenna of the pair; and determining a gas hold-up of the multiphase fluid based at least in part on one or more of the mixture permittivity and the mixture conductivity. In such an example, determining the gas hold-up can be based on the one or more of the mixture permittivity and the mixture conductivity and on the property of the water liquid phase.

As an example, a method can include a water analysis sensor that is or includes a microwave reflection probe for transmitting a signal to multiphase fluid flowing in a near-wall portion of a conduit and receiving at least a portion of the signal as reflected by the multiphase fluid. Such a method can include determining at least one of a mixture conductivity or a mixture permittivity relative to a near-wall portion of the multiphase fluid, where the determining a property of a liquid water phase of the multiphase fluid is determined based on one or more of a mixture conductivity and a mixture permittivity relative to the near-wall portion of the multiphase fluid.

As an example, a method can include receiving flowing multiphase fluid in a conduit that includes a flow restriction and, for example, acquiring measurements that includes acquiring a measurement using a differential pressure sensor to measure a pressure difference between a first location upstream of the flow restriction and a second location in the flow restriction or downstream of the flow restriction. In such an example, the method can include determining a flow rate of at least one phase of the multiphase fluid based on at least one property of a liquid water phase, on gas hold-up, and on the differential pressure measurement.

As an example, a method can include determining a mixture permittivity based on at least a portion of a signal received by a second antenna of a pair of antennas by performing at least one of: determining a resonance frequency of the at least a portion of the signal; and determining a phase shift and/or amplitude attenuation at one or more frequencies of the at least a portion of the signal.

As an example, a method can include determining a type of flow of multiphase fluid, where the type of flow can be an oil continuous flow, a water continuous flow, or a gas continuous mist flow. In such an example, the method can include determining at least one property of a liquid water phase based on a water analysis sensor measurement, for example, by determining at least a water liquid ratio and determining a gas hold-up that is based on the water liquid ratio, a mixture permittivity and/or a mixture conductivity and a dielectric mixing model, where the dielectric mixing model is selected based on the determined type of flow. In such an example, determining the gas hold-up can include, when the type of flow is determined to be a mist flow, determining a liquid mixture permittivity based on at least one of an oil continuous dielectric mixing model and a water continuous dielectric mixing model using the water liquid ratio, and determining a gas hold-up based on a gas continuous dielectric mixing model using the liquid mixture permittivity and the mist flow mixture permittivity. In such an example, the determining the liquid mixture permittivity can include: determining the liquid mixture permittivity based on a water continuous dielectric mixing model for a water liquid ratio over a first threshold; determining the liquid mixture permittivity based on an oil continuous dielectric mixing model for a water liquid ratio under a second threshold; and determining the liquid mixture permittivity based on a combination of a first liquid mixture permittivity determined based on an oil continuous dielectric mixing model and a second liquid mixture permittivity based on a water continuous dielectric mixing model when the water liquid ratio is between the first and second threshold.

As an example, a method can include receiving a multiphase fluid that is a two-phase fluid as flowing in a conduit where the two-phase fluid includes a water liquid phase and a steam phase as a gaseous phase. In such an example, the method can include determining at least one property of the water liquid phase by, for example, determining water salinity. In such an example, the method can include determining gas hold-up of the multiphase fluid based at least on a mixture permittivity or a mixture conductivity and on the water salinity. In such an example, the determining the gas hold-up of the multiphase fluid can be based at least on the mixture permittivity or the mixture conductivity and on at least one of a water permittivity and a water conductivity derived from the water salinity.

As an example, one or more computer-readable storage media can include processor-executable instructions executable by a processor to instruct a system to: acquire measurements with respective sensors relative to one or more properties of multiphase fluid flowing in a conduit, where the sensors include at least a water analysis sensor and a pair of microwave antennas, where the pair of microwave antennas includes a first antenna for transmitting a signal and a second antenna for receiving at least a portion of the signal; determine at least a property of a liquid water phase of the multiphase fluid based on an acquired water analysis sensor measurement, where the at least a property includes one or more of a water salinity and a water liquid ratio; determine one or more of a mixture permittivity and a mixture conductivity of the multiphase fluid based on the at least a portion of the signal received by the second antenna of the pair; and determine a gas hold-up of the multiphase fluid based at least in part on one or more of the mixture permittivity and the mixture conductivity.

In an embodiment, a computer program product is provided that includes computer-executable instructions to instruct a computing system to perform a method such as, for example, the method 800 of FIG. 8 or any of the methods described in the foregoing example embodiments.

A computer-readable storage medium (or computer-readable storage media) is non-transitory, not a signal and not a carrier wave. Rather, a computer-readable storage medium is a physical device that can be considered to be circuitry or hardware.

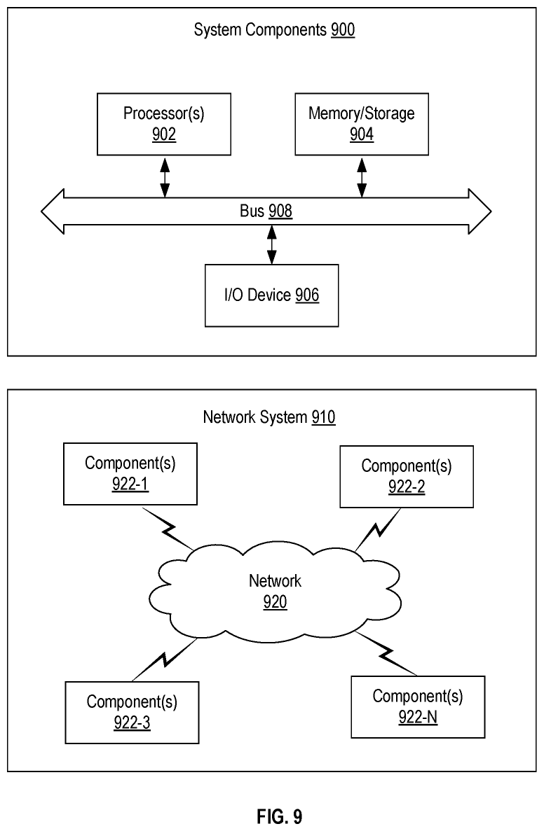

FIG. 9 shows components of an example of a computing system 900 and an example of a networked system 910 with a network 920. The system 900 includes one or more processors 902, memory and/or storage components 904, one or more input and/or output devices 906 and a bus 908. In an example embodiment, instructions may be stored in one or more computer-readable media (e.g., memory/storage components 904). Such instructions may be read by one or more processors (e.g., the processor(s) 902) via a communication bus (e.g., the bus 908), which may be wired or wireless. The one or more processors may execute such instructions to implement (wholly or in part) one or more attributes (e.g., as part of a method). A user may view output from and interact with a process via an I/O device (e.g., the device 906). In an example embodiment, a computer-readable medium may be a storage component such as a physical memory storage device, for example, a chip, a chip on a package, a memory card, etc. (e.g., a computer-readable storage medium). As an example, the processing system 114 of FIG. 1 can include one or more features of the computing system 900 and/or the network system 910.

In an example embodiment, components may be distributed, such as in the network system 910. The network system 910 includes components 922-1, 922-2, 922-3, . . . 922-N. For example, the components 922-1 may include the processor(s) 902 while the component(s) 922-3 may include memory accessible by the processor(s) 902. Further, the component(s) 922-2 may include an I/O device for display and optionally interaction with a method. The network may be or include the Internet, an intranet, a cellular network, a satellite network, etc.

As an example, a processing system can be a device that may be, for example, a mobile device that includes one or more network interfaces for communication of information. For example, a mobile device may include a wireless network interface (e.g., operable via IEEE 802.11, ETSI GSM, BLUETOOTH, satellite, etc.). As an example, a mobile device may include components such as a main processor, memory, a display, display graphics circuitry (e.g., optionally including touch and gesture circuitry), a SIM slot, audio/video circuitry, motion processing circuitry (e.g., accelerometer, gyroscope), wireless LAN circuitry, smart card circuitry, transmitter circuitry, GPS circuitry, and a battery. As an example, a mobile device may be configured as a cell phone, a tablet, etc. As an example, a method may be implemented (e.g., wholly or in part) using a mobile device. As an example, a system may include one or more mobile devices.

As an example, a system may be a distributed environment, for example, a so-called “cloud” environment where various devices, components, etc. interact for purposes of data storage, communications, computing, etc. As an example, a device or a system may include one or more components for communication of information via one or more of the Internet (e.g., where communication occurs via one or more Internet protocols), a cellular network, a satellite network, etc. As an example, a method may be implemented in a distributed environment (e.g., wholly or in part as a cloud-based service).

As an example, information may be input from a display (e.g., consider a touchscreen), output to a display or both. As an example, information may be output to a projector, a laser device, a printer, etc. such that the information may be viewed. As an example, information may be output stereographically or holographically. As to a printer, consider a 2D or a 3D printer. As an example, a 3D printer may include one or more substances that can be output to construct a 3D object. For example, data may be provided to a 3D printer to construct a 3D representation of a subterranean formation. As an example, layers may be constructed in 3D (e.g., horizons, etc.), geobodies constructed in 3D, etc. As an example, holes, fractures, etc., may be constructed in 3D (e.g., as positive structures, as negative structures, etc.).

Although only a few example embodiments have been described in detail above, those skilled in the art will readily appreciate that many modifications are possible in the example embodiments. Accordingly, all such modifications are intended to be included within the scope of this disclosure as defined in the following claims. In the claims, means-plus-function clauses are intended to cover the structures described herein as performing the recited function and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures.

What is claimed is:

1. A system comprising:
a conduit;
a water analysis sensor arranged on the conduit that measures at least a property of multiphase fluid in the conduit, wherein the multiphase fluid comprises at least a gaseous phase and a liquid water phase;
at least a pair of microwave antennas, wherein the pair of microwave antennas comprises a first antenna that transmits a signal into the multiphase fluid in the conduit and a second antenna that receives at least a portion of the signal as transmitted into the multiphase fluid in the conduit; and
a processing system that comprises a processor, memory accessible to the processor, and processor-executable instructions stored in the memory and executable by the processor to cause the processing system to:
determine at least a property of the liquid water phase based on output of the water analysis sensor, wherein the at least a property comprises one or more of a water salinity and a water liquid ratio,
determine one or more of a mixture permittivity and a mixture conductivity of the multiphase fluid based on output of the second antenna, and
determine a gas hold-up of the multiphase fluid based at least in part on one or more of the mixture permittivity and the mixture conductivity.

2. The system of claim 1, wherein the gas hold-up is determined based at least in part on the at least a property of the water liquid phase and one or more of the mixture permittivity and the mixture conductivity.

3. The system of claim 1, wherein the water analysis sensor comprises a microwave reflection probe that transmits a microwave signal to the multiphase fluid in a near-wall portion of the conduit and that receives the at least a portion of the microwave signal as a reflected portion by the multiphase fluid and wherein the processing system comprises processor-executable instructions stored in the memory and executable by the processor to cause the processing system to determine one or more of a near-wall mixture conductivity and a near-wall mixture permittivity of a near-wall portion of the multiphase fluid and wherein the at least a property of the liquid water phase is determined based at least in part on one or more of the near-wall mixture conductivity and the near-wall mixture permittivity.

4. The system of claim 1, further comprising a flow restriction in the conduit and a differential pressure sensor that measures a pressure difference between a first location upstream of the flow restriction and a second location in the flow restriction or downstream of the flow restriction.

5. The system of claim 4, wherein the flow restriction is a Venturi.

6. The system of claim 5, wherein the pair of microwave antennas are positioned in a throat of the Venturi.

7. The system of claim 4, wherein the differential pressure sensor is a multi-variable transmitter that acquires at least one measurement of one or more of a temperature and a pressure at one of the first and second locations.

8. The system of claim 1, wherein the processing system comprises processor-executable instructions stored in the memory and executable by the processor to cause the processing system to determine a flow rate of at least one phase of the multiphase fluid based at least in part on the at least a property of the liquid water phase, the gas hold-up and the differential pressure measurement.

9. The system of claim 1, wherein the mixture permittivity of the multiphase fluid based on output of the second antenna is based at least in part on one or more of a determination of a resonance frequency of the at least a portion of the signal received by the second antenna and a determination of a phase shift and/or amplitude attenuation at one or more frequencies of the at least a portion of the signal received by the second antenna.

10. The system of claim 1, wherein the at least one pair of antennas comprises two pairs of antennas.

11. The system of claim 1, wherein the water analysis sensor comprises a microwave reflection probe that transmits a microwave signal to the multiphase fluid and wherein the microwave reflection probe operates as the second antenna of the pair of antennas.

12. The system of claim 1, wherein the multiphase fluid is a three-phase fluid that comprises the liquid water phase, the gaseous phase and a liquid oil phase.

13. The system of claim 12, wherein the processing system comprises processor-executable instructions stored in the memory and executable by the processor to cause the processing system to determine a type of flow of the multiphase fluid as being an oil continuous flow, a water continuous flow, or a gas continuous mist flow.

14. The system of claim 13, wherein the processing system comprises processor-executable instructions stored in the memory and executable by the processor to cause the processing system to determine at least the water liquid ratio based at least in part on the output of the water analysis sensor and to determine the gas hold-up based at least in part on the water liquid ratio, the mixture permittivity and/or the mixture conductivity and a dielectric mixing model, wherein the dielectric mixing model is selected based on the type of flow.

15. The system of claim 14, wherein, for determining the gas hold-up when the type of flow is determined to be a mist flow, the processing system comprises processor-executable instructions stored in the memory and executable by the processor to cause the processing system to: determine a liquid mixture permittivity based on one or more of an oil continuous dielectric mixing model and a water continuous dielectric mixing model using the water liquid ratio; and determine a gas hold-up based on a gas continuous dielectric mixing model using the liquid mixture permittivity and a mist flow mixture permittivity.

16. The system of claim 15, wherein the dielectric mixing model is a Ramu-Rao or Maxwell-Garnett dielectric mixing model.

17. The system of claim 15, wherein the processing system comprises processor-executable instructions stored in the memory and executable by the processor to cause the processing system to: determine the liquid mixture permittivity based on a water continuous dielectric mixing model for a water liquid ratio over a first threshold; determine the liquid mixture permittivity based on an oil continuous dielectric mixing model for a water liquid ratio under a second threshold; and determine the liquid mixture permittivity based on a combination of a first liquid mixture permittivity determined based on an oil continuous dielectric mixing model and a second liquid mixture permittivity based on a water continuous dielectric mixing model when the water liquid ratio is between the first and second threshold.

18. The system of claim 1, wherein the multiphase fluid is a two-phase fluid that comprises the water liquid phase and a steam phase as the gaseous phase and wherein the processing system comprises processor-executable instructions stored in the memory and executable by the processor to cause the processing system to determine the gas hold-up of the multiphase fluid based at least in part on the mixture permittivity or the mixture conductivity and on the water salinity.

19. A method comprising:
receiving flowing multiphase fluid in a conduit;
acquiring measurements with respective sensors relative to one or more properties of the multiphase fluid, wherein the sensors comprise at least a water analysis sensor and a pair of microwave antennas, wherein the pair of microwave antennas comprises a first antenna for transmitting a signal and a second antenna for receiving at least a portion of the signal;
determining at least a property of a liquid water phase of the multiphase fluid based on an acquired water analysis sensor measurement, wherein the at least a property comprises one or more of a water salinity and a water liquid ratio;
determining one or more of a mixture permittivity and a mixture conductivity of the multiphase fluid based on the at least a portion of the signal received by the second antenna of the pair; and
determining a gas hold-up of the multiphase fluid based at least in part on one or more of the mixture permittivity and the mixture conductivity.

20. One or more computer-readable storage media comprising processor-executable instructions executable by a processor to instruct a system to:
acquire measurements with respective sensors relative to one or more properties of multiphase fluid flowing in a conduit, wherein the sensors comprise at least a water analysis sensor and a pair of microwave antennas, wherein the pair of microwave antennas comprises a first antenna for transmitting a signal and a second antenna for receiving at least a portion of the signal;
determine at least a property of a liquid water phase of the multiphase fluid based on an acquired water analysis sensor measurement, wherein the property comprises one or more of a water salinity and a water liquid ratio;
determine one or more of a mixture permittivity and a mixture conductivity of the multiphase fluid based on the at least a portion of the signal received by the second antenna of the pair; and
determine a gas hold-up of the multiphase fluid based at least in part on one or more of the mixture permittivity and the mixture conductivity.
